(19) United States
(12) Patent Application Publication
(10) Pub. No.: US 2026/0307663 A1
(43) Pub. Date: Oct. 8, 2026

(54) APPARATUS AND METHOD FOR CONFIGURING VEHICLE ENVIRONMENT

(71) Applicants: Hyundai Motor Company, Seoul (KR); Kia Corporation, Seoul (KR)

(72) Inventors: Jungmin Jo, Hwaseong-si (KR); Dongsuk Lee, Hwaseong-si (KR)

(73) Assignees: Hyundai Motor Company, Seoul (KR); Kia Corporation, Seoul (KR)

(21) Appl. No.: 19/325,259

(22) Filed: Sep. 10, 2025

(30) Foreign Application Priority Data
Apr. 4, 2025 (KR) 10-2025-0044034

(51) Int. Cl. B60W 50/00; A61B 5/0205; A61B 5/024; A61B 5/145; B60H 1/00; B60H 3/00; B60K 35/10; B60Q 3/74; B60Q 3/80; B60W 10/30; B60W 50/14

(52) U.S. Cl. CPC B60W 50/0098; A61B 5/02055; A61B 5/14542; B60H 1/00742; B60H 3/0035; B60K 35/10; B60Q 3/74; B60Q 3/80; B60W 10/30; B60W 50/14; A61B 5/02438; B60Q 2900/30; B60W 2050/146; B60W 2510/06; B60W 2540/22; B60W 2540/221; B60W 2540/229; B60W 2556/45; B60W 2710/06; B60W 2710/30; B60W 2756/10

(57) ABSTRACT

In an apparatus and method for configuring a vehicle environment, the apparatus for configuring a vehicle environment may include a communication unit configured to communicate with a wearable device and receive information about a user from the wearable device, and a processor unit configured to, based on the information about the user, control vehicle settings including settings for one or more of engine start, an air conditioning system, a seat mode, an audio playback mode, a mood lamp color, a fragrance emission, or external communication.

### CROSS-REFERENCE TO RELATED APPLICATION

This present application claims the benefit of priority to Korean Patent Application No. 10-2025-0044034, filed on Apr. 4, 2025, the entire contents of which is incorporated herein for all purposes by this reference.

### BACKGROUND OF THE PRESENT DISCLOSURE

### Field of the Present Disclosure

The present disclosure relates to an apparatus and method for configuring a vehicle environment, and more particularly, to an apparatus and method for configuring a vehicle environment by controlling an in-vehicle environment based on information received from a wearable device.

### Description of Related Art

As interest in health and wellness has increased in recent years, a number of companies have been developing wearable devices such as smartwatches and smart rings. These wearable devices may monitor various physical data of a user in real time and provide personalized services to the user based on the monitored data.

The usage of wearable devices has been steadily increasing, and the number of smartwatches sold is expected to reach approximately 230.3 million units by 2025. The wearable device may measure and provide various types of health-related information, including blood oxygen saturation, electrocardiogram (ECG), high and low heart rate, alerts for irregular heartbeats, sleep monitoring, menstrual cycle tracking, and body temperature measurement.

Accordingly, the wearable device becomes a useful tool for identifying a health condition of the user and implementing various application services based on the identified condition. Such technological advancements are opening up possibilities for new application areas that utilize data from wearable devices.

However, most existing wearable devices focus primarily on monitoring and analyzing the user's own health data, and therefore have limitations in linking the data obtained from the wearable device to actual environmental control through connectivity with other devices or systems. In particular, a vehicle lacks sufficient technology for automatically configuring an optimal environment based on the condition of the user, resulting in inconvenience for users who are required to manually control the environment.

The matters described in this Background section are only for enhancement of understanding of the background of the present disclosure, and should not be taken as acknowledgement that they correspond to related art already known to those skilled in the art.

### BRIEF SUMMARY

The following summary presents a simplified summary of certain features. The summary is not an extensive overview and is not intended to identify key or critical elements.

The present disclosure is directed to providing an apparatus and method for configuring a vehicle environment based on a user's physical condition using data received from a wearable device.

Furthermore, the present disclosure is directed to providing an apparatus and method for configuring a vehicle environment that may reduce user inconvenience in configuring the vehicle environment by providing integrated functionalities for improving a condition of a user.

Furthermore, the present disclosure is directed to providing an apparatus and method for configuring a vehicle environment that may detect emergency situations, such as abnormal body conditions, and prevent accidents through preemptive responses.

Aspects of the present disclosure are not limited to those mentioned above, and other aspects and advantages not mentioned above will be understood from the following description, and become more apparent from the exemplary embodiments of the present disclosure. Moreover, aspects of the present disclosure may be realized by the means and combinations thereof indicated in claims.

An aspect of the present disclosure may provide an apparatus for configuring a vehicle environment, including a communication unit configured to communicate with a wearable device and receive information about a user from the wearable device, and a processor unit configured to, based on the information about the user, control vehicle settings including settings for one or more of engine start, an air conditioning system, a seat mode, an audio playback mode, a mood lamp color, a fragrance emission, or external communication.

The information about the user may include biometric signal information of the user, and the processor unit may be configured to control one or more of the air conditioning system, the seat mode, or the mood lamp color based on the biometric signal information of the user.

The biometric signal information of the user may include body temperature information of the user, and the processor unit may be configured to set the air conditioning system and the seat mode to a cooling mode, and the mood lamp color to blue, based on the body temperature of the user being equal to or greater than a first set value, set the air conditioning system and the seat mode to a heating mode, and the mood lamp color to yellow, based on the body temperature of the user being equal to or lower than a second set value, and set the air conditioning system and the seat mode to a ventilation mode, and the mood lamp color to white, based on the body temperature of the user being lower than the first set value and higher than the second set value.

Another aspect of the present disclosure may provide a method for configuring a vehicle environment, including communicating with a wearable device and receiving information about a user from the wearable device, and controlling, based on the information about the user, vehicle settings including settings for one or more of engine start, an air conditioning system, a seat mode, an audio playback mode, a mood lamp color, a fragrance emission, or external communication.

The information about the user may include biometric signal information of the user, and the controlling may include controlling one or more of the air conditioning system, the seat mode, or the mood lamp color based on the biometric signal information of the user.

The biometric signal information of the user may include body temperature information of the user, and the controlling may include setting the air conditioning system and the seat mode to a cooling mode, and the mood lamp color to blue, based on the body temperature of the user being equal to or greater than a first set value, setting the air conditioning system and the seat mode to a heating mode, and the mood lamp color to yellow, based on the body temperature of the user being equal to or lower than a second set value; and setting the air conditioning system and the seat mode to a ventilation mode, and the mood lamp color to white, based on the body temperature of the user being lower than the first set value and higher than the second set value.

According to various aspects of the present disclosure, a vehicle environment may be configured based on a user's physical condition using data received from a wearable device.

Furthermore, according to various aspects of the present disclosure, by providing integrated functionalities for improving a condition of the user, user inconvenience in configuring a vehicle environment may be reduced.

Furthermore, according to various aspects of the present disclosure, when an emergency situation, such as an abnormal body condition of a user, is detected, preemptive action may be taken to prevent accidents.

### BRIEF DESCRIPTION OF THE DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1 is a block diagram of a vehicle environment configuring apparatus according to an exemplary embodiment of the present disclosure.

FIG. 2 is a flowchart of a vehicle environment configuring method according to an exemplary embodiment of the present disclosure.

FIG. 3 is a diagram illustrating classification of a temperature of a wearable device user into high, normal, and low body temperature according to an exemplary embodiment of the present disclosure.

FIG. 4 is a table illustrating vehicle settings based on classified body temperatures according to an exemplary embodiment of the present disclosure.

FIG. 5 shows an example computing system

### DETAILED DESCRIPTION

Hereinafter, various exemplary embodiments included in the present specification will be described in detail with reference to the accompanying drawings. Like reference numerals designate like elements, and redundant descriptions thereof will be omitted. Furthermore, such as “module” and a “unit”, suffixes for components used in the following description are provided or mixed and used by considering easiness in preparing a specification and do not include a meaning or role distinguished from each other in themselves. Furthermore, in describing an exemplary embodiment disclosed in the present specification, if it is determined that a detailed description of a related art incorporated herein unnecessarily obscure the gist of the exemplary embodiment of the present disclosure, the detailed description thereof will be omitted. Furthermore, it should be understood that the appended drawings are intended only to help understand embodiments disclosed in the present specification and do not limit the technical principles and scope of the present disclosure; rather, it should be understood that the appended drawings include all of the modifications, equivalents or substitutes described by the technical principles and belonging to the technical scope of the present disclosure.

Unless otherwise defined, all technical and scientific terms used in the present disclosure have the same meanings as commonly understood by those skilled in the art of the present application. The terms “include,” “comprise,” and any variations thereof used in the specification of the present application are intended to encompass non-exclusive inclusion.

Although the terms first, second, and the like, may be used herein to describe various elements, these elements should not be limited by these terms. These terms are generally only used to distinguish one element from another. When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present.

The term “unit” or “module” used in the present specification signifies one unit that processes at least one function or operation, and may be realized by hardware, software, or a combination thereof. The operations of the method or the functions described in connection with the forms disclosed herein may be embodied directly in a hardware or a software module executed by a processor, or in a combination thereof.

For purposes of the present application and the claims, using the exemplary phrase “at least one of: A; B; or C” or “at least one of A, B, or C,” the phrase means “at least one A, or at least one B, or at least one C, or any combination of at least one A, at least one B, and at least one C. Furthermore, exemplary phrases, such as “A, B, and C”, “A, B, or C”, “at least one of A, B, and C”, “at least one of A, B, or C”, etc. As used herein may mean each listed item or all possible combinations of the listed items. For example, “at least one of A or B” may refer to (1) at least one A; (2) at least one B; or (3) at least one A and at least one B.

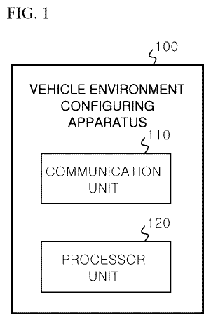

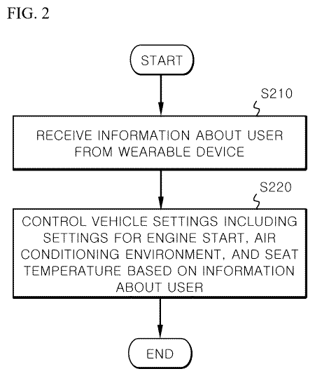

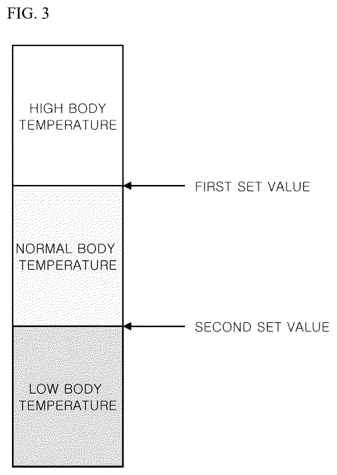

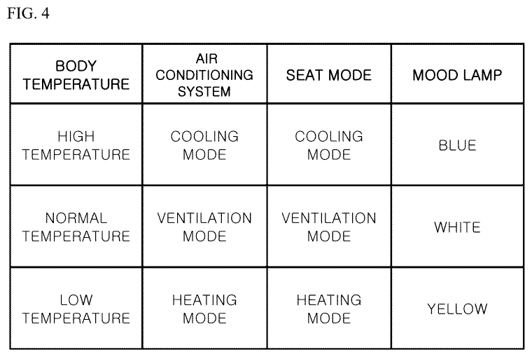

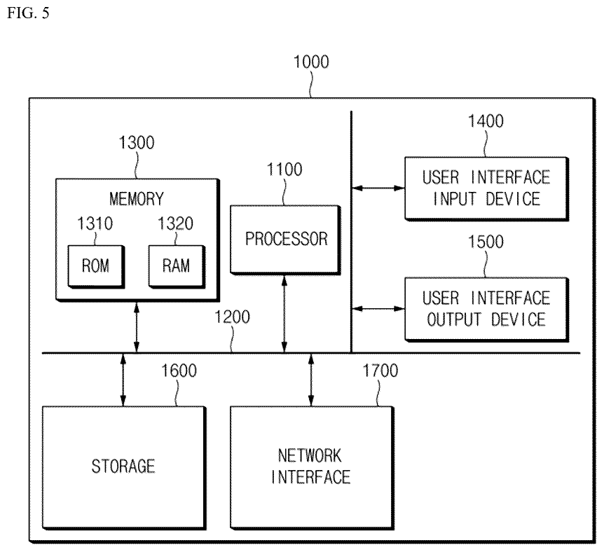

Hereinafter, a vehicle environment configuring apparatus and method according to an exemplary embodiment of the present disclosure will be described in detail with reference to FIG. 1, FIG. 2, FIG. 3, FIG. 4 and FIG. 5.

FIG. 1 is a block diagram of a vehicle environment configuring apparatus according to an exemplary embodiment of the present disclosure, and FIG. 2 is a flowchart of a vehicle environment configuring method according to an exemplary embodiment of the present disclosure.

Referring to FIG. 1, a vehicle environment configuring apparatus according to an exemplary embodiment of the present disclosure may include a communication unit 110, a processor unit 120, and other components.

The communication unit 110 may communicate with a wearable device and may receive information about a user from the wearable device (see S210 of FIG. 2).

For example, the communication unit 110 may communicate with the wearable device, such as a smartwatch, fitness band, smart ring, or smart glasses, which may measure information about a user, and may receive the information about the user. The information about the user may include, for example, biometric signal information, location information, condition information, and input information entered by the user into the wearable device.

The processor unit 120 may, based on the information about the user, control vehicle settings including settings for one or more of engine start, an air conditioning system, a seat mode, an audio playback mode, a mood lamp color, a fragrance emission, or external communication (see S220 of FIG. 2).

The processor unit 120 may analyze the received information about the user to determine the current condition of the user and may configure a vehicle environment by controlling vehicle settings to improve the user's condition.

The processor unit 120 may be configured to determine whether the user is approaching the vehicle based on location information of the user and may start the engine of the vehicle accordingly.

The wearable device may be configured to determine in real time whether the user is approaching the vicinity of the vehicle by use of location tracking functions such as a Global Positioning System (GPS) module or Bluetooth. The processor unit 120 may receive location information from the wearable device, and when it is determined that the user has entered a predetermined range (for example, within 50 to 100 meters of the vehicle), may initiate engine start of the vehicle so that other vehicle settings such as air conditioning control, seat mode setting, and audio mode switching may be activated. At the instant time, door unlocking of the vehicle may also be performed.

Furthermore, the processor unit 120 may be configured for controlling one or more of the air conditioning system, seat mode, or mood lamp based on biometric signal information of the user.

FIG. 3 is a diagram illustrating classification of a temperature of a wearable device user into high, normal, and low body temperature according to an exemplary embodiment of the present disclosure.

Referring to FIG. 3, the processor unit 120 may classify the user's body temperature as high when the temperature is measured to be equal to or greater than a first set value, for example, 38° C. The processor unit 120 may classify the temperature as normal when the temperature is lower than the first set value but higher than a second set value, for example, 36° C. The processor unit 120 may classify the temperature as low when the temperature is measured to be equal to or lower than the second set value. The processor unit 120 may configure the vehicle environment by controlling the air conditioning system, seat mode, and mood lamp based on each classified body temperature.

FIG. 4 is a table illustrating vehicle settings based on classified body temperatures according to an exemplary embodiment of the present disclosure.

Referring to FIG. 4, when the body temperature of the user of the wearable device falls under the high temperature classification, a cooling mode of the air conditioning system may be activated, and the seat may also be set to a cooling mode to help lower the user's body temperature. Furthermore, the mood lamp may be set to a cool blue-toned color, lowering the perceived temperature and visually generating a cooler atmosphere inside the vehicle.

Meanwhile, when the body temperature of the user of the wearable device falls under the low temperature classification, the processor unit 120 may activate a heating mode of the air conditioning system and set the seat to a heating mode to help raise the user's body temperature. Furthermore, the mood lamp may be set to a warm yellow-toned color, increasing the perceived temperature and visually generating a warmer atmosphere inside the vehicle.

When the body temperature of the user of the wearable device falls under the normal temperature classification, the air conditioning system and the seat may be set to a ventilation mode to maintain the user's body temperature and to generate a comfortable environment. The mood lamp may be set to a white-toned color to visually generate a neutral interior and to produce a bright and clean atmosphere.

Furthermore, the wearable device may measure various types of biometric signal information, such as sleep duration, step count, heart rate, calories burned, and respiration rate. The processor unit 120 may configure the vehicle environment by controlling vehicle settings based on the biometric signal information.

For example, when the sleep duration is shorter than a predetermined threshold, the processor unit 120 may be configured to determine that the user is in a fatigued state. Accordingly, the processor unit 120 may configure the vehicle environment to assist with the user's alertness by setting the air conditioning system to a cool temperature and adjusting the color of the mood lamp to a yellow tone. When the user is determined to be in a drowsy state based on additional analysis of information such as heart rate and respiration rate, the user's concentration may be enhanced by outputting an alert sound through the vehicle audio system or by activating a subtle seat vibration function.

Furthermore, the processor unit 120 may analyze data such as step count, heart rate, calories burned, and respiration rate to determine that the user has engaged in physical activity prior to entering the vehicle. For example, the processor unit 120 may be configured to determine that the user has entered the vehicle immediately after exercising based on detection of a high heart rate and a high amount of calories burned, and may adjust the internal temperature to a comfortable level while activating a ventilation mode to help the user cool down rapidly. Furthermore, the mood lamp may be set to a blue tone to generate a calm atmosphere, or the massage function of the seat may be activated to help the user recover from fatigue.

The processor unit 120 may also control one or more of an audio playback mode or fragrance emission based on the condition information of the user.

The condition information may include a stress index, sleep duration, and a post-exercise recovery indicator measured by the wearable device. The processor unit 120 may configure the vehicle environment by analyzing the psychological state or fatigue level of the user based on the condition information.

When the stress index is equal to or greater than a predetermined value (e.g., 70), when the sleep duration is less than or equal to a predetermined threshold (e.g., 7 hours), or when the post-exercise recovery indicator is equal to or greater than a predetermined threshold (e.g., 50), the processor unit 120 may be configured to determine that the user is in a fatigued or tense state, and may play preconfigured audio and emit a preconfigured fragrance to respond accordingly. The processor unit 120 may cause the vehicle audio system to play music that helps promote psychological stability and recovery, such as soft classical music, calm jazz music, or natural ambient sounds. The processor unit 120 may also adjust the vehicle environment by causing a fragrance dispenser to release scents such as lavender, sandalwood, or peppermint, promoting physical and psychological recovery.

Meanwhile, when the stress index is below a predetermined value (e.g., 70), the sleep duration exceeds a predetermined threshold (e.g., 7 hours), and the post-exercise recovery indicator is below a predetermined recovery threshold (e.g., 50), the processor unit 120 may be configured to determine that the user is in a physically and mentally stable state with sufficient energy, and may configure the vehicle environment to respond accordingly.

It should be understood that the exemplary values described above are provided merely for illustrative purposes. For example, the stress index and the post-exercise recovery indicator may each be represented on a scale of 0 to 100, where values such as “70” or “50” are exemplary reference points that may vary depending on the particular algorithm, user calibration, or commercially available measurement device. Likewise, the sleep duration threshold of “7 hours” is also provided as an exemplary reference point, which may be adjusted based on user characteristics, health guidelines, or application requirements.

In such a case, the processor unit 120 may cause the vehicle audio system to play lively music to help maintain the user's vitality and positive state. The processor unit 120 may automatically recommend and cause the system to play music with an active mood, such as upbeat move music or high-tempo jazz. The processor unit 120 may also adjust the vehicle environment by causing a fragrance dispenser to release refreshing scents, such as citrus-based or fresh herbal fragrances, maintaining a pleasant and energetic atmosphere inside the vehicle.

In the instant case, the processor unit 120 may receive information about the user from the wearable device in real time and may configure the vehicle environment based on the received information.

The processor unit 120 may provide control information for vehicle settings to one or more of a vehicle infotainment display or an instrument cluster.

For example, when the user's sleep duration is determined to be insufficient and the user is analyzed to be in a drowsy state, the processor unit 120 may display a pop-up message, such as “The vehicle environment has been configured to improve alertness in response to insufficient sleep,” on the infotainment display or instrument cluster to notify the user that the current air conditioning settings and seat ventilation settings have been adjusted to mitigate drowsiness. When the user's body temperature is measured to be higher than a predetermined temperature and condition improvement is needed, the processor unit 120 may display a message such as “A cool vehicle environment has been configured based on your high body temperature to improve your condition,” to notify the user that the cooling mode of the air conditioning system and seat cooling have been activated. Conversely, when the user's body temperature is lower than a predetermined temperature, the processor unit 120 may display a message such as “A comfortable vehicle environment has been configured based on your low body temperature to improve your condition,” to notify the user that the heating mode of the air conditioning system and seat heating have been activated.

When it is determined that the user's body temperature has decreased while the vehicle is in motion, the processor unit 120 may display a message such as “A comfortable vehicle environment is being configured based on your low body temperature detected while driving to improve your condition.” The message may notify the user, along with the body temperature information, that the heating mode of the air conditioning system and seat heating are about to be adjusted, and may prompt the user to select whether to proceed.

The wearable device may also provide control information for vehicle settings through its display or voice output.

Meanwhile, when the user's biometric signal deviates from a predetermined range, the processor unit 120 may perform external communication.

For example, when the processor unit 120 detects that the heart rate measured by the wearable device suddenly drops below 40 beats per minute or rapidly rises above 180 beats per minute, when a high heart rate of 150 beats per minute or more persists for a predetermined time period (e.g., 10 minutes), when a low heart rate of 50 beats per minute or less persists for a predetermined time period (e.g., 10 minutes), when the oxygen saturation remains below a predetermined level (e.g., 90%) for a predetermined time period (e.g., 20 minutes), or when the oxygen saturation level decreases rapidly, a message such as “A health abnormality has been detected. Would you like to initiate communication with a designated center?” may be displayed on the vehicle display or instrument cluster. Based on the user's approval, the processor unit 120 may perform automatic communication with a designated center, such as a fire station or emergency response center.

The processor unit 120 may continuously monitor changes in the biometric signals of the user, and when an abnormal state is not resolved and persists for a predetermined period of time, may cause the communication unit 110 to immediately place an emergency call or transmit an emergency message.

The processor unit 120 may also receive user inputs from the wearable device, such as activation status information of the vehicle settings, activation scheduling information of the vehicle settings, and control information for each of the vehicle settings, and may apply the user inputs to vehicle control operations.

For example, when the user's body temperature is measured to be higher than a predetermined temperature, the processor unit 120 may display a message on the wearable device, such as “Would you like to pre-cool the vehicle for your arrival?” and may receive user confirmation to activate the corresponding function.

When the user set a schedule through the wearable device application to, for example, initiate engine start at 7 a.m. every day, set the air conditioning temperature to 23 degrees Celsius, and activate the seat heater at level 1, the processor unit 120 may automatically activate the corresponding vehicle settings according to the schedule.

When the user registers a schedule in the wearable device, such as “Activate seat ventilation at level 2 around 9 p.m. every day,” considering the time of return home after exercise, the processor unit 120 may automatically operate the cooling and seat ventilation functions at the scheduled time so that the user may enjoy a cool environment immediately upon entering the vehicle.

When the user registers a schedule through the wearable device, such as “Change the mood lamp color to party mode at 6 p.m. every Friday” to switch the in-vehicle atmosphere after work, the processor unit 120 may recognize the scheduled information, and at the specified time, automatically change the mood lamp color and activate the audio playback mode to generate an enjoyable atmosphere.

Meanwhile, the processor unit 120 may learn user-profile information such as the user's driving history, vehicle boarding habits, and trends in biometric signal changes. The processor unit 120 may, based on learned user profile information, prioritize and activate vehicle settings that are frequently selected at the time of engine start, and may adjust their priority order.

For example, the processor unit 120 may learn that the user habitually activates cooling mode and seat ventilation immediately after initiating engine start during work commutes. Based on the present pattern, the processor unit 120 automatically prioritizes these functions at the time of engine startup, allowing the user to experience the exemplary environment without manual operation. Based on weekend evening driving patterns indicating short trips and frequent changes in the audio playback mode, the processor unit 120 may prioritize the audio playback mode at the time of engine start and may activate the mood lamp color to generate a comfortable in-vehicle environment.

FIG. 5 shows an example computing system (e.g., a computing device of a vehicle or any other apparatus). One or more controllers, processors, etc. described herein, such as the communication unit 110 and the processor unit 120, or any other components and devices disclosed herein, may be implemented by or in the computing system as shown in FIG. 5.

A computing system 1000 may include at least one processor 1100, memory 1300, a user interface input device 1400, a user interface output device 1500, a storage 1600, and a network interface 1700, which are connected with each other via a bus 1200.

The processor 1100 may be a central processing unit (CPU) or a semiconductor device that processes instructions stored in the memory 1300 and/or the storage 1600. Each of the memory 1300 and the storage 1600 may include various types of volatile or nonvolatile storage media. For example, the memory 1300 may include a read-only memory (ROM) and a random-access memory (RAM).

Communication interface(s) (also referred to as communication device(s), communicator(s), communication module(s), communication unit(s), etc.), such as the network interface 1700, may allow software and/or data to be transferred between a device and one or more external devices, and/or between one or more components of a device. Communication interface(s) may include a receiver, a transmitter, a transceiver, a modem, a network interface and/or adapter (such as an Ethernet adapter), a radio transceiver, an antenna, a communication port, a Personal Computer Memory Card International Association (PCMCIA) slot and card, or the like. Software and data transferred via communication interface(s) may be in the form of signals, which may be electronic, electromagnetic, optical, infrared, or other signals capable of being received by communication interface(s). These signals may be provided to communication interface(s) via a communication path of a device, which may be implemented using, for example, wire or cable, fiber optics, a cellular link, a radio frequency (RF) link and/or other communications channels. Communication interface(s) may communicate using one or more communication protocols, such as Ethernet, Wi-Fi, near-field communication (NFC), Infrared Data Association (IrDA), Bluetooth, Bluetooth low energy (BLE), Zigbee, Long-Term Evolution (LTE), 5G New Radio (NR), vehicle-to-everything (V2X), a controller area network (CAN), or a local interconnect network (LIN), etc.

Accordingly, the operations of the method or algorithm described in connection with example example(s) disclosed in the specification may be directly implemented with a hardware module, a software module, or a combination of the hardware module and the software module, which is executed by the processor 1100. The software module may reside on a storage medium (e.g., the memory 1300 and/or the storage 1600) such as RAM, a flash memory, ROM, an erasable and programmable ROM (EPROM), an electrically EPROM (EEPROM), a register, a hard disk drive, a removable disc, or a compact disc-ROM (CD-ROM).

The storage medium may be coupled to the processor 1100. The processor 1100 may read out information from the storage medium and may write information in the storage medium. Alternatively, the storage medium may be integrated with the processor 1100. The processor and storage medium may be implemented with an application specific integrated circuit (ASIC). The ASIC may be provided in a user terminal. Alternatively, the processor and storage medium may be implemented with separate components in the user terminal.

As used in the present disclosure (especially in the appended claims), the terms “a/an” and “the” include both singular and plural references, unless the context clearly states otherwise. Also, it should be understood that any numerical range recited in the present disclosure is intended to include all sub-ranges subsumed therein (unless expressly indicated otherwise) and accordingly, the disclosed numeral ranges include every individual value between the minimum and maximum values of the numeral ranges.

The steps forming the method according to an exemplary embodiment of the present disclosure may be performed in an appropriate order unless a specific order is described or otherwise specified. That is, the present disclosure is not necessarily limited to the order in which the steps are recited. All examples described in the present disclosure or the terms indicative thereof (“for example”, “such as”) are merely to describe the present disclosure in greater detail. Therefore, it should be understood that the scope of the present disclosure is not limited to the example embodiments described above or by the use of such terms unless limited by the appended claims. Also, it should be apparent to those skilled in the art that various modifications, combinations, and alternations may be made depending on design conditions and factors within the scope of the appended claims or equivalents thereof.

The present disclosure is thus not limited to the example embodiments described above, and rather intended to include the following appended claims, and all modifications, equivalents, and alternatives falling within the spirit and scope of the following claims.

What is claimed is:

1. An apparatus for configuring a vehicle environment, the apparatus comprising:
a communication unit configured to communicate with a wearable device and receive information about a user from the wearable device; and
a processor configured to, based on the information about the user, control vehicle settings comprising settings for one or more of engine start, an air conditioning system, a seat mode, an audio playback mode, a mood lamp color, a fragrance emission, or external communication.

2. The apparatus of claim 1,
wherein the information about the user comprises biometric signal information of the user, and
wherein the processor is further configured to control one or more of the air conditioning system, the seat mode, or the mood lamp color based on the biometric signal information of the user.

3. The apparatus of claim 2,
wherein the biometric signal information of the user comprises body temperature information of the user, and
wherein the processor is further configured to:
set the air conditioning system and the seat mode to a cooling mode, and the mood lamp color to blue, based on a body temperature of the user being equal to or greater than a first set value;
set the air conditioning system and the seat mode to a heating mode, and the mood lamp color to yellow, based on the body temperature of the user being equal to or lower than a second set value; and
set the air conditioning system and the seat mode to a ventilation mode, and the mood lamp color to white, based on the body temperature of the user being lower than the first set value and higher than the second set value.

4. The apparatus of claim 1, wherein the information about the user comprises condition information comprising one or more of a stress index, sleep duration, or a post-exercise recovery indicator, and the processor is further configured to control one or more of the audio playback mode or the fragrance emission based on the condition information.

5. The apparatus of claim 4, wherein the processor is further configured to play preconfigured audio and emit a preconfigured fragrance based on the stress index being equal to or greater than a predetermined index, the sleep duration being less than or equal to a predetermined time period, or the post-exercise recovery indicator being equal to or greater than a predetermined recovery threshold.

6. The apparatus of claim 1, wherein the information about the user comprises heart rate information and blood oxygen saturation information of the user, and the processor is further configured to cause the external communication to be performed based on a heart rate or a blood oxygen saturation of the user deviating from a predetermined range.

7. The apparatus of claim 1, wherein the information about the user comprises input information entered by the user into the wearable device, the input information comprises one or more of activation status information of the vehicle settings, activation scheduling information of the vehicle settings, or control information for each of the vehicle settings, and the processor is further configured to control the vehicle settings based on the input information.

8. The apparatus of claim 1, wherein the processor is further configured to display control information of the vehicle settings on one or more of an infotainment display or an instrument cluster of the vehicle.

9. The apparatus of claim 1, wherein the information about the user comprises location information of the user, and the processor is further configured to turn on an engine of the vehicle and control the vehicle settings based on the engine being off and the user being located within a predetermined distance from the vehicle.

10. A method for configuring a vehicle environment, the method comprising:
communicating, by a processor, with a wearable device and receiving information about a user from the wearable device; and
controlling, by the processor, based on the information about the user, vehicle settings comprising settings for one or more of engine start, an air conditioning system, a seat mode, an audio playback mode, a mood lamp color, a fragrance emission, or external communication.

11. The method of claim 10,
wherein the information about the user comprises biometric signal information of the user, and
wherein the controlling comprises controlling one or more of the air conditioning system, the seat mode, or the mood lamp color based on the biometric signal information of the user.

12. The method of claim 11, wherein the biometric signal information of the user comprises body temperature information of the user, and the controlling comprises:
setting the air conditioning system and the seat mode to a cooling mode, and the mood lamp color to blue, based on a body temperature of the user being equal to or greater than a first set value;
setting the air conditioning system and the seat mode to a heating mode, and the mood lamp color to yellow, based on the body temperature of the user being equal to or lower than a second set value; and
setting the air conditioning system and the seat mode to a ventilation mode, and the mood lamp color to white, based on the body temperature of the user being lower than the first set value and higher than the second set value.

13. The method of claim 10,
wherein the information about the user comprises condition information including one or more of a stress index, sleep duration, or a post-exercise recovery indicator, and
wherein the controlling comprises controlling one or more of the audio playback mode or the fragrance emission based on the condition information.

14. The method of claim 13, wherein the controlling comprises playing preconfigured audio and emitting a preconfigured fragrance based on the stress index being equal to or greater than a predetermined index, the sleep duration being less than or equal to a predetermined time period, or the post-exercise recovery indicator being equal to or greater than a predetermined recovery threshold.

15. The method of claim 10,
wherein the information about the user comprises heart rate information and blood oxygen saturation information of the user, and
wherein the controlling comprises causing the external communication to be performed based on a heart rate or a blood oxygen saturation of the user deviating from a predetermined range.

16. The method of claim 10,
wherein the information about the user comprises input information entered by the user into the wearable device, the input information comprises one or more of activation status information of the vehicle settings, activation scheduling information of the vehicle settings, or control information for each of the vehicle settings, and
wherein the controlling comprises controlling the vehicle settings based on the input information.

17. The method of claim 10, wherein the controlling comprises displaying control information of the vehicle settings on one or more of an infotainment display or an instrument cluster of the vehicle.

18. The method of claim 10,
wherein the information about the user comprises location information of the user, and
wherein the controlling comprises turning on an engine of the vehicle and controlling the vehicle settings based on the engine being off and the user being located within a predetermined distance from the vehicle.
